(19) United States
(12) Patent Application Publication
(10) Pub. No.: US 2022/0143111 A1
(43) Pub. Date: May 12, 2022

(54) METHOD OF TREATING AND/OR PREVENTING ASTHMA, ASTHMA EXACERBATIONS, ALLERGIC ASTHMA AND/OR ASSOCIATED CONDITIONS WITH MICROBIOTA RELATED TO RESPIRATORY DISORDERS

(71) Applicants: OM PHARMA SA, Meyrin (CH); Arizona Board of Regents on behalf of the Univeristy of Arizona, Tucson, AZ (US)

(72) Inventors: Donata VERCELLI, TUCSON, AZ (US); Christian PASQUALI, Prangins (CH); Vadim PIVNIOUK, Tucson, AZ (US); Fernando MARTINEZ, TUCSON, AZ (US); Jacques BAUER, St-Prex (CH)

(21) Appl. No.: 17/439,112

(22) PCT Filed: Sep. 13, 2019
(86) PCT No.: PCT/EP2019/074562

§ 371 Date: Sep. 14, 2021

(30) Foreign Application Priority Data
Mar. 14, 2019 (EP) 19162717.3
Mar. 14, 2019 (RU) 2019107206

(51) Int. Cl. A61K 35/744; A61K 35/741; A61P 11/06

(52) U.S. Cl. CPC A61K 35/744; A61P 11/06; A61K 35/741

(57) ABSTRACT

The present invention relates to novel methods and routes of administration of specific bacterial extracts obtainable by alkaline lysis of Gram positive or Gram negative bacterial species and delivery devices for treating and/or preventing asthma, asthma exacerbation, allergic asthma conditions and/or wheezing associated symptoms and/or associated with microbiota related to respiratory disorders in a subject in need thereof comprising administering a therapeutically effective amount of bacterial extract obtainable from Gram positive or Gram negative bacterial species wherein said bacterial extract is obtainable by alkaline lysis.

### FIELD OF THE INVENTION

The present invention relates to novel methods and routes of administration of specific bacterial extracts obtainable by alkaline lysis of Gram positive or Gram negative bacterial species and delivery devices for treating and/or preventing asthma, asthma exacerbations, allergic asthma and/or wheezing associated symptoms and/or associated conditions with microbiota related to respiratory disorders in human subjects, reorganization of a protective microbiota in asthmatic subjects as well as reorganization of microbiota towards protective immunity in healthy subjects.

### BACKGROUND OF THE INVENTION

Chronic airway diseases such as asthma, allergic asthma and other wheezing disorders are one of the major healthcare issues particularly in children. Asthma is an inflammatory disease of the airways in the lungs and bronchial tubes, resulting in temporarily inflamed, constricted and narrowing airways. Asthma may be caused by primary infections, by allergens or irritants that are inhaled into the lungs. Symptoms include difficulty in breathing, excessive mucus and phlegm production, wheezing, cough and tightness in the chest. The underlying cause of asthma is a complex product of genetic and environmental factors resulting in significant heterogeneity of the disease. The prevalence of asthma has dramatically increased in westernized countries in recent decades and this is likely due to changing environmental exposures.

Also, recent evidence supports a role of the microbiota in the development of asthma and wheezing disorders, suggesting that changes in the microbiota are linked to chronic airway disease symptoms, and in particular to asthma and wheezing asthma. The hypothesis is that early life exposures may disrupt the composition of the microbiota, consequently promoting immune dysregulation in the form of hypersensitivity disorders.

A healthy microbiome provides the host with multiple benefits, including resistance to colonization by a broad spectrum of pathogens, essential nutrient biosynthesis and absorption, as well as immune defense and stimulation that maintains a healthy gut epithelium and appropriately controlled systemic immunity. Thus, the lung airway and intestinal microbiota plays a significant role in the pathogenesis of many diseases and disorders, including a variety of pathogenic infections of the gut.

In settings of imbalance, microbiota functions can be lost or deranged, resulting in increased susceptibility to pathogens, altered metabolic profiles, or induction of proinflammatory signals that can result in local or systemic inflammation or autoimmunity. In particular, microbiota changes have been linked to several chronic airway diseases and symptoms such as asthma and wheezing. Consequently, patients become more susceptible to pathogenic infections when the normal intestinal microbiota has been disturbed due to use of broad-spectrum antibiotics. Many of these diseases and disorders are chronic conditions that significantly decrease a patient's quality of life and can be ultimately fatal.

The present invention is based on the surprising discovery that the administration of specific bacterial extracts obtainable by alkaline lysis of Gram positive or Gram negative bacterial species provides an effective and safe approach for preventing allergic inflammation in the airways, while restoring healthy microbiome in a host suffering from asthma and allergy-induced exacerbations apparently associated with imbalanced microbiota.

### SUMMARY OF THE INVENTION

The present invention relates to novel routes of administration and dosages of bacterial extract for use in methods of treating and/or preventing asthma, asthma exacerbations, allergic asthma conditions, wheezing as well as associated microbiota-related disorders in a subject in need thereof. Different experiments using similar or various airway inflammation inducers have been described in the figures.

The present invention also relates to a method of monitoring a treatment or prevention of a disease or abnormality or imbalanced microbiome in a test subject: (a) treating the test subject with therapeutically effective amount of purified bacterial extract obtainable by alkaline lysis of Gram positive and/or Gram negative bacterial species (b) analyzing a signature of the microbiome of the test subject; and (c) comparing said microbiome signature or pattern of the test subject with the microbiome signature or pattern of a healthy subject, wherein an increase in the similarity of the microbiome signature of the test subject with the microbiome signature of the healthy subject following the treatment with therapeutically effective amount of stable purified bacterial extract as compared to the similarity of the microbiome signature of the test subject with the microbiome signature of the healthy subject prior to the treatment is indicative of an effective treatment. Different figures aiming at describing gene correlation and changes, as well as discrimination, correlation and taxonomic changes with the test subjects have been also presented.

### BRIEF DESCRIPTION OF THE FIGURES

FIG. 1: shows the protocol used in the first study to assess the effects of BV bacterial extract (BV) in a classic allergen-driven mouse model of experimental asthma using BALB/c strain mice.

FIG. 2: (A) shows the effects of BV on bronchoalveolar lavage (BAL) eosinophilia expressed in percentage of cell content in the BAL fluid obtained in the second study. The study was performed using the protocol of FIG. 1 except for BV dose. Significant inhibition of eosinophilia increase was obtained using the 1 mg dose of BV administered 14 times. (B) shows another representative example of efficacy by BV demonstrating a dose-dependent decrease of BAL eosinophilia in an asthma model using different doses.

FIG. 3: (A) shows the effects of BV on bronchoalveolar lavage (BAL) eosinophilia as in FIG. 2 but originating from a different experiment and where eosinophilia content from BAL was expressed as total cell number. (B) shows how BV suppresses the cardinal molecular phenotypes of asthma-induced lung inflammation: BAL protein levels of IL-5 and IL-13 in pg/mL.

FIG. 4: shows a representative example of the functional efficacy correlation depicted in FIGS. 2 and 3 illustrated here with lung function as a readout. Airway hyper responsiveness (AHR) was significantly prevented by BV with best efficacy at 1 mg dose in all tested points from previous studies. Significant reduction of AHR was expressed relative to OVA.

FIG. 5: shows an additional example demonstrating how BV suppresses the cardinal cellular and molecular phenotypes of asthma-induced lung inflammation, here shown with a strongly significant decrease of mucus-secreting goblet cells detected by PAS* staining in BV-treated animals. *PAS=periodic acid Schiff stain.

FIG. 6: shows the protocol used to assess the protective effect of BV in the allergic asthma model driven by Alternaria alternata.

FIG. 7: shows a representative example of the protective effect of BV in the Alternaria allergic asthma model following intranasal administration. Monitoring of the efficacy by BV was based on BAL differentials cell content and demonstrated approximatively 40% inhibition of eosinophilia expressed here as the percentage of cells in BAL fluid obtained following the experimental scheme described in Example 5.

FIG. 8: shows the effects of i.n. BV in the Alternaria model: Respiratory System Resistance (Rrs)—max response (represents total airway resistance). Rrs response was significantly prevented by BV with best efficacy at 1 mg dose in all tested points from previous studies. Significant reduction of AHR was expressed relative to Alternaria.

FIG. 9: shows the in vitro prevention by BV of stress- and Alternaria-induced impairment of barrier function (measured as TEER, transepithelial electrical resistance) in human bronchial epithelial cells (16HBE14o-cell line).

FIG. 10: shows a shorter protocol of OVA-induced asthma (19 instead of 39 days, and 8 instead of 14 treatments, compared to FIG. 1).

FIG. 11: shows BAL eosinophilia blockade by BV expressed here as percentage (left) as well as cell count (right) but originating from the modified OVA-induced scheme depicted in FIG. 10.

FIG. 12: shows an additional protocol for OVA-induced asthma used for C57 BL6 strain mice and tested with BV as previously using 14 treatments with 1 mg dose/day/mouse delivered intranasally.

FIG. 13: shows that BV bacterial extract induced a protective decrease in eosinophilia as well as a protective increase of neutrophilia in BAL as expressed in cell percentage.

FIG. 14: WGCNA—Identification of gene groups (modules) that are regulated by OVA and/or

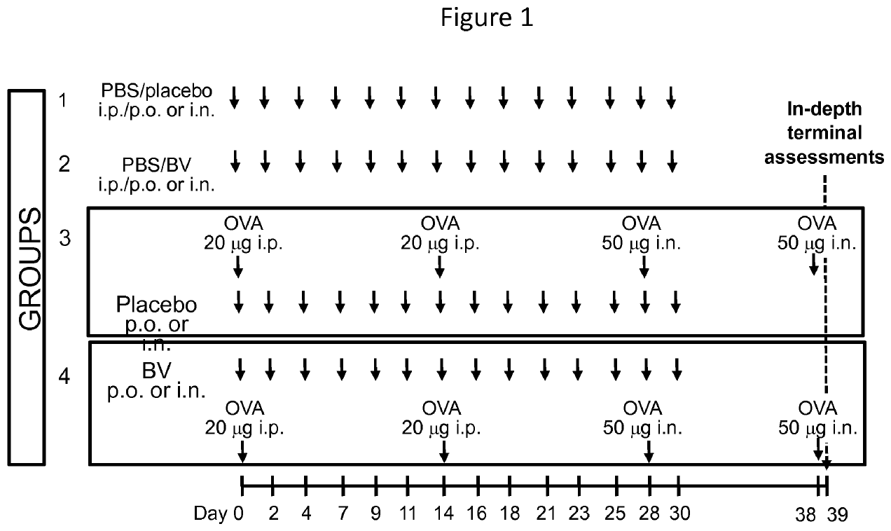

BV and are associated with cardinal asthma phenotypes in the lungs of mice treated as in FIG. 1 and analyzed by RNA-Seq. Each box shows the Pearson's correlation coefficient (r) for the relationship between each module eigengene (the first principal component of the standardized gene expression profiles of the module, i.e., a summary of the standardized module gene expression data) and traits (percent BAL eosinophils, AHR Z-score) and/or comparisons (OVA versus PBS, OVA+BV versus OVA) of interest. Levels of significance are shown as q-values in parentheses within each box. The number of genes within each module is in parentheses next to each module name. The intensity of color reflects the strength of the association.

FIG. 15 Ingenuity Pathway Analysis (IPA) on a core set of genes from WGCNA-identified modules highly associated with eosinophilia, AHR and OVA/BV treatment demonstrates that these genes clustered not only, as expected, in pathways related to AHR and cell movement of eosinophils, but also in pathways related to migration and differentiation of Th2 cells and dendritic cell chemotaxis. The strategy for identifying a core set of genes is shown on the left. The panel on the right shows selected biological functions (rectangles) that are enriched among members of this core gene set (symbols). Genes are annotated with values for log₂ (Fold Change) between OVA+BV and OVA treated mice from differential expression analysis. Enrichment (P-values) and predicted activation values (Z-score) for each pathway are written on each rectangle. Symbol shape and color corresponds to module membership: turquoise: grey: diamonds, brown: grey circles, blue: open circles, and yellow: open squares.

FIG. 16: is a PCoA plot of unweighted UniFrac beta diversity across three experiments (#100, 101, 113) showing clustering of gut microbiota samples from mice treated intranasally with BV (1 or 2.25 mg/mouse×14 treatments) with or without OVA, or OVA alone, or PBS. All treatments were significantly different from one another (q<0.05; PERMANOVA).

FIG. 17: shows on the left taxonomic profiles at the phylum level for all groups and samples shown in FIG. 16, which evidenced differences in communities; and on the right, alpha diversity for all groups and samples shown in FIG. 16, measured by observed ESVs and Shannon Diversity Index, with boxes representing the 25-75% quartiles, and whiskers representing 95% confidence intervals.

FIG. 18: shows in (A) Linear discriminant analysis effect size (LEfSe) of the samples from FIGS. 16 and 17 into two groups: Any BV (BV/OVA and BV only) and No BV (OVA only and PBS only), showing taxa that are enriched in each group, as indicated by color. This analysis revealed differential features at multiple taxonomic levels between, using a cutoff of q<0.05 and LDA score>2. (B) Phylogenetic representation of each of the differential taxa. (g, genus; f, family; o, order; c, class).

### DETAILED DESCRIPTION OF THE INVENTION

The present invention thus provides a method of treating and/or preventing asthma, asthma exacerbations, allergic asthma conditions, wheezing associated symptoms, as well as microbiota associated disorders in a subject in need thereof or at risk of developing such disorders, comprising administering a therapeutically effective amount of BV bacterial extract obtainable by alkaline lysis of Gram positive or Gram negative bacterial species. Said bacterial extract may be administered via oral, intranasal, or intratracheal route. Particularly preferred routes of administration include intranasal and intratracheal routes.

Asthma condition may be steroid resistant asthma, neutrophilic asthma or non-allergic asthma. The allergic disease or disorder may be an eosinophilic disease or disorder, particularly a disease or disorder selected from the group consisting of nodules, eosinophilia, eosinophilic rheumatism, dermatitis and swelling (NERDS).

Intranasal and intratracheal routes of administration of the BV bacterial extract according to the present invention have been showed as being particularly advantageous for use in methods of treating and/or preventing and/or attenuating asthma, asthma exacerbations, allergic asthma conditions, wheezing as well as associated microbiota related respiratory disorders in a subject in need thereof.

Applicants demonstrated in the Examples herein below that administration of BV bacterial extract provided an efficient protection against allergic asthma, at lower dosages and was efficient in conferring a complete protection against allergen-driven airway hyper responsiveness (AHR) and airway inflammation, in providing a complete abrogation of eosinophilia and an increased protective effect of neutrophilia using different lung airway inflammation models. In addition, Applicants showed that compared to per oral, lower dosages of BV bacterial extract via the intranasal or the intratracheal route resulted in a complete abrogation of eosinophilia in the BAL from lung, as well as a significant decrease of several TH2 cytokines levels of which, IL13 and IL5.

In particular, intranasal or intratracheal administrations allow use of lower dosages of BV bacterial extract and even at such lower dosages have been evidenced to induce a significant reorganization of the microbiome content of the gut, and provided a protective effect against allergic asthma and a substantial decrease of the allergic response through changes of the microbiome.

“By bacterial extracts” refer to bacterial extract obtainable by alkaline lysis of one or more pathogens chosen among Moraxella catarrhalis, Haemophilus influenzae, Klebsiella pneumoniae, Staphylococcus aureus, Streptococcus pneumoniae, Streptococcus pyogenes, and/or Streptococcus sanguinis as described in international publication No. WO2008/109669. The process of preparation of the BV bacterial extract prepared by alkaline lysis, preferably at a pH greater than 10 was also described in the international publication No. WO2008/109669. Preferably, said BV bacterial extract may comprise the combination of the all of the above listed pathogens which is marketed under the trademark Broncho-Vaxom® for treating respiratory disorders.

Typically, BV bacterial extracts are prepared by fermentation followed by heat inactivation and alkaline lysis and filtration. Fermentation, alkaline lysis and filtration are now well-known in the art and as described inter alia in international publication WO2008/109669.

Fermentation is generally performed by growing each bacteria strain to a suitable optical density in a culture medium. For each strain, to obtain a sufficient amount of material, the fermentation cultures may be started from a working seed lot followed by inoculation into larger fermentation containers. For example, fermentation may start with a small culture such as 0.1 to 1.0 liter, incubated for about 3 to 6 hours at 30 to 40° C., such as 37° C., to obtain an optical density (OD) at 700 nm of 3.0 to 5.0. After a small-scale culture step, additional cultures in one or a series of larger fermenters may be performed at 30° C. to 40° C. for a duration of 3 hours to 20 hours, such as for 3-10 hours, or 8 hours.

The culture medium is preferably a medium that does not pose a risk of prion-related diseases (i.e., mad cow disease, scrapie, and Creutzfeld-Jacob disease) or other diseases and thus that does not comprise animal-based materials such as serum or meat extracts taken from animals such as cows or sheep or from any other animal that can transmit prion-based diseases. For example, a non-animal medium, such as a vegetable-based medium, such as a soya-based medium, or a synthetic or hemi-synthetic medium, may be used. Alternatively, media using horse serum or media comprising materials taken from animal species that do not transmit prion diseases may be used. The culture medium may also include biological extracts such as yeast extract and horse serum, which also do not pose such disease risks. Supplementary growth factors may also be introduced to enhance the growth of some bacteria species.

After fermentation, the biomass from each bacteria strain or from combined bacteria strains is generally inactivated by a heat treatment, concentrated, and frozen.

Alkaline lysis is used for lysing bacterial cells under basic conditions and is generally performed by using an organic or inorganic base. Alkaline lysis may be performed on a single bacterial biomass or on mixture of bacterial biomass or fermentation batches, under basic conditions, typically with a concentrated solution of hydroxide ions, such as from NaOH. Alkaline lysis may be performed preferably at a pH greater than around 10, with variations of ±0.1 of the pH. Duration of the lysis may be assessed by the skilled person in the art and depends on the initial bacterial biomass amount. Lysis may be performed at temperatures ranging from 30 to 60° C., such as from 30-40° C., or from 35-40° C., such as 37° C. In general, lysis is stopped when bacteria cells appear to all have been disrupted based on a visual observation as this is well-known to the skilled person in the art. When using more than one strain of the same bacterial genus, the strains may be lysed together or separately. The strains may thus be mixed before or after lysis.

The lysates are then purified by centrifugation and/or filtration to remove large cellular debris or any components that are insufficiently degraded, any insoluble or particulate material so as to obtain a soluble bacterial extract. Purification including centrifugation and filtration are well-known in the art to remove particulate matter from the extracts. For example, lysates may be centrifuged at 9000 g, followed by one or more rounds of filtration. Typically, the filtration may comprise the passage of an extract or a mixture of extracts, through one or more filters such as microfilters (i.e., microfiltration) or ultrafilters (i.e., ultrafiltration) which may be repeated in several passes or cycles. For example, successive rounds of filtration on larger pore filters followed by microfiltration using a smaller pore filter may be performed, such as a 0.2 micron filter. Ultrafiltration may also be employed in order to help extract soluble materials from the extract, for example, recirculating the ultrafiltration permeate for further microfiltration. Tangential flow filtration (TFF) method may be used to filter the extracts and to extract solubilized molecules from larger cellular debris. This is well-known in the art and described inter alia by Wayne P. Olson (Separations Technology, Pharmaceutical and Biotechnology Applications, Interpharm Press, Inc., Buffalo Grove, Ill., U.S.A., pp. 126-135).

Up to this date, BV bacterial extract has been administered per oral to the patients for preventing respiratory tract infections in adults and children. Several clinical trials have demonstrated that the enteral administration (per oral) of the BV bacterial extract was capable of preventing allergic asthma and wheezing attacks provoked by acute respiratory tract illnesses in children. Said BV bacterial extracts have been commercialized under the tradename of Broncho-Vaxom®. The BV bacterial extract drug is in solid form, generally a capsule, which is administered to the patients per oral and at dose regimens of one capsule per day of 7 mg of lyophilized bacteria extract for adults treatment, and one capsule per day of 3.5 mg lyophilized bacteria extract for children.

According to a preferred embodiment, the present invention relates to method of treating and/or preventing asthma, asthma exacerbation, allergic asthma conditions, wheezing associated symptoms, microbiota associated disorders in a subject in need thereof comprising administering a therapeutically effective amount of BV bacterial extract via intranasal or intratracheal routes. The present invention also relates to BV bacterial extract for use in a method of treating and/or preventing asthma, asthma exacerbation, allergic asthma conditions, wheezing associated symptoms, microbiota associated disorders in a subject in need thereof wherein said BV bacterial extract is administered via intranasal or intratracheal routes to said subject.

Intranasal or intratracheal administration of BV bacterial extracts are particularly useful for treating and/or preventing asthma, asthma exacerbation, allergic asthma conditions and/or wheezing associated symptoms and/or associated with microbiota related disorders in human subjects, reorganization of a protective microbiota in asthmatic subjects as well as organization of a microbiota towards protective immunity in healthy subjects.

The term “microbiota” refers to a community of commensal, symbiotic, and/or pathogenic microbes. Microbes or micro-organisms live in practically every part of the ecosphere and are found everywhere in and on the human body, including the mucosal linings in nasal passages, oral cavities, vagina, skin, gastrointestinal tract, and the urogenital tract. The term “microbiome” refers to the full collection of microbes and the genetic information of those microbes within a specific body area (the “habitat”) of the host. Each type of microbe may produce a different effect in the context in which the microbe comes to live. The composition of the microbiota that may reside on and within, for example, a mammalian organism may affect immune function, nutrient processing, and other aspects of physiology. The composition of the microbiota may change over time and can be affected by age, diet, antibiotic exposure, and other environmental influences. When different microbial species produce largely the same effect, the microbiota is said to have some functional redundancy. An addition or loss of microbial species that produce similar effects may have little influence on the overall effect that the microbiota has on the physiology of the mammalian system. However, the addition or loss of certain microbial species, even if present in small numbers, may produce a significant effect on mammalian physiology.

The term “microbiome imbalance” or “dysbiosis” refers to the state in which a system has an imbalance in the beneficial and harmful microbes. Dysbiosis can occur when there is a low diversity of beneficial microbial species and/or a lack of functional redundancy of beneficial microbes in the microbiota. When the microbial species that produce what is considered to be a beneficial effect on the system are not at least equal to the microbial species that produce what is considered to be a harmful effect on the system, a microbial imbalance is said to have been created. Typically, in case of dysbiosis, the microbiome signature shows a reorganization of an altered microbiome and an increase of Lactobacillus taxa.

The present invention relates also to a method of treatment and of prevention of such diseases and their proven link to microbiota related disorders in human subjects limited to the reorganization of a protective microbiota in asthmatic subjects as well as its organization towards protective immunity in healthy subjects.

During the past few years a large international effort, called the Human Microbiome Project (HMP) by the National Institutes of Health, and known more broadly as the International Human Microbiome Consortium (IHMC), is aimed at characterizing the microbes living in and on our bodies (see http://hmpdacc.org/data_browser.php). In the large intestine an estimated 100 trillion microorganisms reside that appear to play essential roles in metabolizing food, drugs, and dietary supplements that are not absorbed by the upper gastrointestinal (GI) tract. In addition, some of those microorganisms manufacture essential nutrients and vitamins necessary to sustain health. Such microbial interactions in the intestinal environment exert critical roles in signaling metabolic-, behavior-, and immune-regulatory systems of the human host.

Microbiomes comprise commensal, symbiotic, and pathogenic bacteria, fungi, and viruses, which form an ecological entity and interact with themselves and with their particular host. For a long time, it has been assumed that microbiota colonization is restricted to body surfaces like skin and the gastrointestinal tract. However, it became clear in the recent years that microorganisms reside in nearly every human tissue including the mammary glands, the ovaries, the uterus, the placenta and the lung. Thus the human body is colonized by trillions of microbial inhabitants. They constitute a diverse and individually varying ecological community which in addition changes with age.

Various microbiomes have been directly implicated in the etiopathogenesis of a number of pathological states as diverse as asthma, asthma exacerbation, allergic asthma conditions, and wheezing associated symptoms.

Therefore, BV bacterial extracts as described in the above embodiments are thus beneficial in restoring natural microbiome and particularly useful for treating and/or asthma, asthma exacerbation, allergic asthma conditions, wheezing associated symptoms, microbiota associated disorders.

A therapeutically effective amount of BV bacterial extract provided is such that the signature or pattern of the microbiome of the subject is made more similar to the signature or pattern of the microbiome of a healthy subject, thereby treating and/or preventing any pathological states related to microbiome imbalance. What is considered to be a healthier system may be established or reestablished by limiting the harmful microbes while promoting the development of the beneficial microbes. In this case, the eubiosis state is promoted, wherein the beneficial microbes within a system have a dominant effect because there is a high diversity and/or a functional redundancy of the beneficial microbial species in the system.

BV bacterial extracts may be present in any forms suitable for oral administration, such as for example solid or liquid form, and may be formulated as a pill, a tablet, a film table, a coated tablet, a capsule, syrup, powder and/or deposit.

According to a preferred embodiment, BV bacterial extracts may be present in any forms, either liquid, gas, or solid forms, suitable for intranasal and intratracheal administrations. When BV bacterial extracts are present in liquid or aerosol form, they may be formulated in a spray, droplet, colloidal, mist, nebulae, or in atomized vapor. Alternatively, they may be present in solid form and are then formulated in powders, or crushable tablets.

When the BV bacterial extract preparations are administered via the intranasal route, then they are preferably in a form chosen from an emulsion, suspension, colloidal form, mist, nebulae, atomized vapor or a spray, droplet, colloidal, mist, nebulae, atomized vapor, a nasal tampon, a nasal emulsion, a powder, an ointment, a cream, a lotion, a gel, a paste, a salve, solution, tincture, patch, a bioadhesive strip.

When the BV bacterial extract preparations are administered via the intratracheal route, delivery requires aerosolization of a solid or liquid and delivery of the aerosol to the lungs via the mouth and throat. Particles of the BV bacterial extracts may be administered to the lungs as dry powder aerosols or liquid aerosols. Dry powder aerosols are generally administered to the lungs with dry powder inhaler (DPI) inhalation devices. Dry powder inhalers can include breath actuated dry powder inhalers, such as are described in U.S. Pat. No. 7,434,579. Metered-dose inhalers contain medicament suspended in a propellant, a mixture of propellants, or a mixture of solvents, propellants, and/or other excipients in compact pressurized aerosol dispensers. An MDI product may discharge up to several hundred metered doses of BV bacterial extract. Each actuation may contain from a few micrograms (mcg) up to milligrams (mg) of the active ingredients delivered in a volume typically between 25 and 140 microliters.

Another type of liquid aerosol dispersion device is nebulizer, which uses a jet, a vibrating mesh or other means to aerosolize a suspension containing particles of the BV bacterial extract.

In the preparation of liquid, semi-solid, solid, and spray medicines, BV bacterial extracts may be formulated with any additives such as vehicles, binding agents, perfumes, flavoring agents, sweeteners, colorants, antiseptics, antioxidants, stabilizing agents, and surfactants, if desired.

To prepare the above-mentioned pharmaceutical formulations, BV bacterial extracts may be mixed with a pharmaceutical acceptable carrier, adjuvant and/or excipient, according to conventional pharmaceutical compounding techniques. Pharmaceutically acceptable carriers that can be used in the present compositions encompass any of the standard pharmaceutical carriers, such as a phosphate buffered saline solution, water, and emulsions, such as an oil/water or water/oil emulsion, and various types of wetting agents. The composition can additionally contain solid pharmaceutical excipients such as starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols. For examples of carriers, stabilizers and adjuvants, see Remington's Pharmaceutical Sciences, edited by E. W. Martin (Mack Publishing Company, 18th ed., 1990). The bacterial extract preparations may also include stabilizers and preservatives.

In certain embodiments, it can be desirable to prolong the residence time in the nasal cavity, for example, to enhance absorption. Thus, the BV bacterial extract may optionally be formulated with a bioadhesive polymer, a gum, such as xanthan gum, chitosan (e.g., highly purified cationic polysaccharide), pectin, or any carbohydrate that thickens like a gel or emulsifies when applied to nasal mucosa, a microsphere, such as, starch, albumin, dextran, cyclodextrin, or derivatives thereof, gelatin, a liposome, carbamer, polyvinyl alcohol, alginate, acacia, chitosans and/or cellulose (e.g., methyl or propyl; hydroxyl or carboxy; carboxymethyl or hydroxylpropyl).

Alternatively, BV bacterial extract preparations may be formulated as a crushable tablet. The tablet can be administered whole or lightly crushed, such as with finger pressure, and sprinkled over an appropriate vehicle. The crushable tablet can be prepared using direct compression processes and excipients with care taken in the process to avoid damaging the coating of the individual subunits. Suitable excipients to prepare the crushable tablet include those typically used for chewable tablets including mono- and di-saccharides, sugar polyols, and the like, or a combination thereof. Exemplary excipients include mannitol, sorbitol, xylitol, maltitol, lactose, sucrose, maltose or a combination thereof. Optional pharmaceutical excipients such as diluents, lubricants, glidants, flavorants, colorants, etc. or a combination comprising at least one of the foregoing may also be included in the compression matrix. The crushable tablets can be prepared using methods of tablet manufacturing known in the pharmaceutical art.

BV bacterial extract formulation may be further present in as colloidal form, comprising for example, a metal halide, most preferably silver halide. The BV bacterial extracts and adjuvant may be incorporated within or encapsulated by the colloidal particle. Alternatively, or in addition, one or more bacterial extracts and adjuvant may be attached to a surface of the colloidal particle. For example, proteins readily adsorb or attach to hydrophobic particles via hydrophobic interactions with the particle surface and displace some of the neutral emulsifier.

Suitable dosages of BV bacterial extract will vary depending upon the condition, age and species of the subject and can be readily determined by those skilled in the art. However, according to the present invention, total daily dosages may be in the range of 0.005 to 2 mg, preferably from 0.05 to 1 mg, most preferably from 0.5 to 1 mg and these may be administered as single or divided doses, and in addition, the upper limit can also be exceeded up to 2 mg when this is found to be indicated.

BV bacterial extracts may be administered intranasally by nasal insufflator device, intranasal inhaler, intranasal spray device, atomizer, nasal spray bottle, unit dose container, pump, dropper, squeeze bottle, nebulizer, metered dose inhaler (MDI), pressurized dose inhalers, insufflators, bi-directional devices, dose ampoules, nasal pads, nasal sponges, and nasal capsules.

Also provided is a delivery device for use in a method of treatment and/or prevention of asthma, asthma exacerbation, allergic asthma conditions, wheezing associated symptoms, microbiota associated disorders in a subject in need thereof.

Such delivery devices can be selected from the group comprising of nasal insufflator device, intranasal inhalers, intranasal spray devices, atomizers, nasal spray bottles, unit dose containers, pumps, droppers, squeeze bottles, nebulizers, metered dose inhalers (MDI), pressurized dose inhalers, insufflators, bi-directional devices, dose ampoules, nasal pads, nasal sponges, nasal capsules, and the like.

Nasal sprays may be liquid, solid nasal sprays for administration of as aerosols or in non-aerosol forms. The nasal delivery device can be metered to administer an accurate effective dosage amount of the BV bacterial extract to the nasal cavity. The nasal delivery device can be for single unit delivery or multiple unit delivery. A therapeutically effective amount of the BV bacterial as defined above invention may be delivered through a tube, a catheter, a syringe, a packtail, a pledget, a nasal tampon or by submucosal infusion as described in US Patent Publications US 2009/0326275, 2009/0291894, 2009/0281522 and 2009/0317377.

When intranasal or intratracheal administration is performed with aerosols, aerosol sprays may be generated for example from pressurized container with a suitable propellant such as, dichlorodifluoromethane, trichlorofluoromethane, dichlorotetrafluoroethane, hydrocarbons, compressed air, nitrogen, carbon dioxide, or other suitable gas. The dosage unit can be determined by providing a valve to deliver a metered amount. Pump spray dispensers can dispense a metered dose or a dose having a specific particle or droplet size. As used herein, the term “aerosol” may refer to a suspension or dispersion of either liquid droplets or solid powder in air (or in a gas). Liquid droplets may be formed from solutions, suspensions and dispersions of drug in a liquid, such as water or a non-aqueous solvent. The aerosol may be insufflated or inhaled through the nose. Aerosols may be produced in any suitable device, such as an MDI, a nebulizer, or a mist sprayer.

Typically, aerosol may be insufflated using a suitable mechanical apparatus. In some embodiments, the apparatus may include a reservoir and sprayer, which is a device adapted to expel the pharmaceutical dose in the form of a spray. Several doses of the BV bacterial extract to be administered may be contained within the reservoir, optionally in a liquid solution or suspension or in a solid particulate formulation, such as a solid particulate mixture.

Alternatively, nebulizer devices may be used. These devices produce a stream of high velocity air that causes BV bacterial extract to be administered in the form of liquid to spray as a mist, such as micronized particles, wherein 90% or more of the particles have a diameter of less than about 10.

Another delivery device for intranasal or intratracheal administration of BV bacterial extracts according to the present invention may be DPI devices which typically administer a therapeutically effective amount in the form of a free-flowing powder that can be dispersed in a patient's airstream during inspiration. DPI devices which use an external energy source may also be used in the present invention. In order to achieve a free-flowing powder, the BV bacterial extract may be formulated with a suitable excipient, such as for example lactose. A dry powder formulation can be made, for example, by combining dry lactose having a particle size between about 1 μm and 100 μm with micronized particles of the BV bacterial extract and dry blending. Alternatively, the BV bacterial extract may be formulated without excipients. The formulation is loaded into a dry powder dispenser or into inhalation cartridges or capsules for use with a dry powder delivery device. Examples of DPI devices provided commercially include Diskhaler (GlaxoSmith line, Research Triangle Park, N.C.) (U.S. Pat. No. 5,035,237); Diskus (U.S. Pat. No. 6,378,519; Turbuhaler (U.S. Pat. No. 4,524,769); and Rotahaler (U.S. Pat. No. 4,353,365). Further examples of suitable DPI devices are further described in U.S. Pat. Nos. 5,415,162; 5,239,993; and 5,715,810.

MDI devices typically discharge a therapeutically effective amount of the BV bacterial extract using compressed propellant gas. Formulations for MDI administration include a solution or suspension of active ingredient in a liquefied propellant. The formulation is loaded into an aerosol canister, which forms a portion of an MDI device. Examples of propellants include hydrofluoroalklanes (HFA), such as 1,1,1,2-tetrafluoroethane (HFA 134a) and 1,1,1,2,3,3,3-heptafluoro-n-propane, (HFA 227), and chlorofluorocarbons, such as CCl₃F. Additional components of HFA formulations for MDI administration include co-solvents, such as ethanol, pentane, water; and surfactants, such as sorbitan trioleate, oleic acid, lecithin, and glycerin. MDI devices have been described inter alia in U.S. Pat. No. 5,225,183, and international publication WO92/22286.

When the BV bacterial extracts are in the form of powders, they may be administered via the intranasal or intratracheal route according to the present invention using nasal insufflators. Typically, they may be absorbed to a solid surface, for example, a carrier and are thus delivered to the nasal cavity as a powder in a form such as microspheres. The powder or microspheres may be stored in a container of the insufflator and then may be administered in a dry, air-dispensable form. Alternatively, the powder or microspheres may be filled into a capsule, such as a gelatin capsule, or other single dose unit adapted for nasal administration.

When the BV bacterial extracts of the present invention are delivered through a nasal spray applicator, they may be placed in an intranasal spray-dosing device or atomizer and may be applied by spraying it into the nostrils of a subject for delivery to the mucous membrane of the nostrils. For an intranasal spray, an application of up to about 200 microliters, from about 50 to about 150 microliters, or from about 75 to about 120 microliters may be applied. One or more nostrils may be dosed and application may occur as often as desired or as often as is necessary. A first dose in microliters may be administered into the nostril. After an appropriate time period when the amount of liquid has been absorbed, a second dose may be administered sequentially into the same nostril or may be administered into a different nostril. Additional metered sprays may be applied to alternating nostrils until the full target therapeutic dose has been administered to the patient. There may be a time increment of from several seconds to 5 minutes, preferably about 10 seconds to about 1 minute, between applications of benzodiazepine drug to the same nostril. This allows time for the drug to cross the nasal mucosa and enter the blood stream. Multiple applications of metered sprays to each nostril, optionally separated by a time interval, allows administration of a full therapeutic dose in increments small enough to permit full absorption of the composition into the blood stream and avoid loss of drug down the back of the throat.

BV bacterial extracts may further comprise adjuvants, pharmaceutically acceptable solvents, and permeation enhancers. For example, for intranasal delivery, the permeation enhancer can enhance the permeation of composition through the nasal mucosa. In some embodiments, compounds containing one or more than one hydroxyl group may be used as permeation enhancers. Some of these hydroxyl group-containing compounds can also serve as solvents in the composition. Non-limiting examples of hydroxyl group-containing compounds that may be used as permeation enhancers include alcohols (such as ethanol), diols (such as propylene glycol also known as 1,2-propanediol; 1,3-propanediol; butylene glycol including 1,3-butanediol, 1,2-butanediol, 2,3-butanediol, and 1,4 butanediol; hexylene glycol; dipropylene glycol, 1,5-pentanediol, 1,2-pentanediol, 1,8-octanediol, etohexadiol, p-menthane-3,8 diol, 2-methyl-2,4-pentanediol), triols (such as glycerin), polyols (such as suitable polymers containing multiple hydroxyl groups, including polyethylene glycols or PEGs, polypropylene glycols, polysorbates, and sorbitan esters; and suitable sugar alcohols), cyclitols (such as pinitol, inositol), cyclic diols (such as cyclohexane diol), aromatic diols (such as hydroquinone, bisphenol A, resorcinol and catechol).

One of ordinary skill in the art would recognize that the instant teachings would also be applicable to other permeation enhancers. These may include simple aliphatic, unsaturated or saturated esters. Non-limiting examples of such esters include isopropyl myristate, myristyl myristate, octyl palmitate, and the like. Non-limiting examples other permeation enhancers include alcohols (e.g., short- and long-chain alcohols), polyalcohols, amines and amides, urea, amino acids and their esters, amides, azone or pyrrolidone and its derivatives, terpenes, fatty acids and their esters, macrocyclic compounds, sulfoxides, tensides, benzyldimethylammonium chloride, cetyl trimethyl ammonium bromide, cineole, cocamidopropyl betaine, cocamidopropyl hydroxysultaine, dodecyl pyridinium chloride, dodecylamine, hexadecyl trimethylammoniopropane sulfonate, limonene, linoleic acid (OA), linolenic acid (LA), menthol, methyl laurate, methylpyrolidone, N-decyl-2-pyrrolidone, NLS, nicotine sulfate, nonyl-1,3-dioxolane, octyl trimethylammonium bromide, oleyl betaine, PP, polyethyleneglycol dodecyl ether, polyoxyethelene sorbitan monolaurate (Tween 20, or Polysorbate 20), SLA, sodium oleate, sodium lauryl sulfate, sodium octyl sulfate (SOS), sorbitan monolaurate (S20), tetracaine, and Triton X-100.

Examples of pharmaceutically acceptable solvents that may be used in the present invention may be found in reference books such as the Handbook of Pharmaceutical Excipients (Fifth Edition, Pharmaceutical Press, London and American Pharmacists Association, Washington, 2006). Non-limiting examples of pharmaceutically acceptable solvents that may be used in the present composition include, but are not limited to, propylene glycol, such as 1,2-dihydroxypropane, 2-hydroxypropanol, methyl ethylene glycol, methyl glycol or propane-1,2-diol), ethanol, methanol, propanol, isopropanol, butanol, glycerol, polyethylene glycol (PEG), glycol, Cremophor EL or any forms of polyethoxylated castor oil, dipropylene glycol, dimethyl isosorbide, propylene carbonate, N-methylpyrrolidone, glycofurol, tetraethyleneglycol, propylene glycol fatty acid esters, and mixtures thereof.

The present invention further provides kits comprising BV bacterial extracts as described herein above or a delivery device for inclusion of BV bacterial extract and a package insert incorporating manual instructions for usage.

### First Series of Experiments: Asthma Protection

### Example 1

### Effects of Various Dosages of BV Bacterial Extracts on OVA-Induced Asthma Models

Four mice/group where 6 doses of BV bacterial extract (or BV herein after) were tested in two separate experiments (study 1 and 2) using intranasal route upon anesthesia:
Study 1: 5-50-1000 microgram/treatment×14 treatments.
Study 2: 1-10-100-1000-2250 microgram/treatment of BV×14 treatments.

Regimen was chosen from previous studies performed during the setting up of the ovalbumin-induced (OVA) asthma model using different course with Amish dust (Stein et al, N Engl J Med 2016; 375:411-421). Doses of BV originated from previous optimized studies using viral infection models and extended in this experimental scheme up to 1 and 2.2 mg/dose.

To this end, Phase 1 designed dose-response experiments in which OVA-immunized mice were treated via intranasal/inhalation (i.n.) routes every 2-3 days from day 0 to day 32 (14 times in total) with increasing doses of BV bacterial extract (1, 5, 10, 50, 100 or 1000 microgram/mouse/treatment). The 1 mg/mouse dose was proposed in an exploratory capacity. In Phase 2, the same protocol was adopted to further test the BV concentration that had exhibited optimal effects (1000 microgram/mouse/treatment) and a higher dose (2250 microgram/mouse/treatment) was added to assess whether a plateau of maximal protection could be reached.

OVA-Induced Asthma Animal Model

7-8-week-old male BALB/c mice were purchased from Envigo (United States) and maintained under specific-pathogen free conditions at the BIOS Institute animal facility. Animals were fed a standard hypoallergenic diet.

In the scheme example from FIG. 1, BV bacterial extract concentrate was diluted in 0.9% saline. The OVA model was adapted from the Stein M et al. Reference. Briefly, BV bacterial extract (1, 5, 10, 50, 100, 1000 microgram/mouse/treatment in 25 μl (microliters) of saline) was instilled intranasally (i.n.) under light isoflurane anesthesia evenly into the two nostrils every 2-3 days (14 times total) from day 0 to day 32 into 7-8 week old BALB/c mice (Envigo) that were sensitized intra-peritoneally (i.p.) with ovalbumin (OVA: grade V, Sigma, 20 microgram)-Alum (Pierce) at day 0 and 14, and challenged i.n. with OVA (50 microgram) at day 28 and 38. A group of mice received saline at the time of treatment, sensitization and challenge. In exp. 3, a group of mice was sham treated.

Terminal assessments at day 39 included using the non-exhaustive readouts: 1) invasive measurements of AHR; 2) broncho-alveolar lavage (BAL) cellularity with differentials; 3) levels of lung cytokine RNA and protein; 4) analysis of selected lung cell populations; 5) measurement of serum OVA-specific IgE.

In the initial exploratory experiments (Study 1 and study 2), airway resistance in response to increasing concentrations of nebulized methacholine (0-40 and 0-60 mg/ml) was assessed in animals anesthetized with ketamine and xylazine (100 and 10 mg/kg, respectively) and paralyzed with pancuronium bromide (4 μg/g). The trachea was dissected free and cannulated with a 20-gauge cannula (BD) kept in place with a single tie suture. Mice were then connected to a small ventilator (FlexiVent FX, SCIREQ, Inc.) and ventilated with a tidal volume of 10 mL/kg, inspiratory/expiratory ratio of 66.67%, respiratory rate of 150/min and maximum pressure of 30 cm H2O. In the final experiment with the optimal BV bacterial extract concentration (1,000 microgram/mouse/treatment, Study 3), airway resistance was measured in response to increasing doses of acetylcholine (0-1 microgram/mouse) administered intravenously. BAL was obtained by delivering cold 1% BSA in PBS (2 mL) into the airway via a tracheal cannula and gently aspirating the fluid. Cells were counted using a Countess II FL automated cell counter (Thermo Fisher Scientific) and differentials were determined by an operator blinded to mouse ID/grouping after staining with Hema 3 (Fischer) and examining at least 400 cells/slide. BAL cytokines (IL-5, IL-13, IL-17) were measured by ELISA (R&D). Statistical analysis:

Statistical differences in all parameters were assessed using an unpaired, two-tailed Student's t test. p values<0.05 were statistically significant.

### Example 2

Illustrative example of the effects of intranasal administration of BV bacterial extract in an OVA model of Asthma.

Monitoring of efficacy based on BAL eosinophilia expressed as percentage following the experimental scheme described in Example 1. To assess the efficacy of BV bacterial extract in reducing eosinophilia associated with the protection from lung airway hyper responsiveness (AHR), two separated experiments were conducted with various doses of BV. Best efficacy in reducing eosinophilia (Y axis) was reached with significant inhibition at a dose of 1 mg of BV. The complete absence of eosinophilia effect in the placebo group irrespective of the dose used confirms also the safe use of BV in non-diseased animals. Intranasal/inhalation delivery of BV suppresses the cardinal cellular phenotypes of asthma-induced lung inflammation.

### Example 3

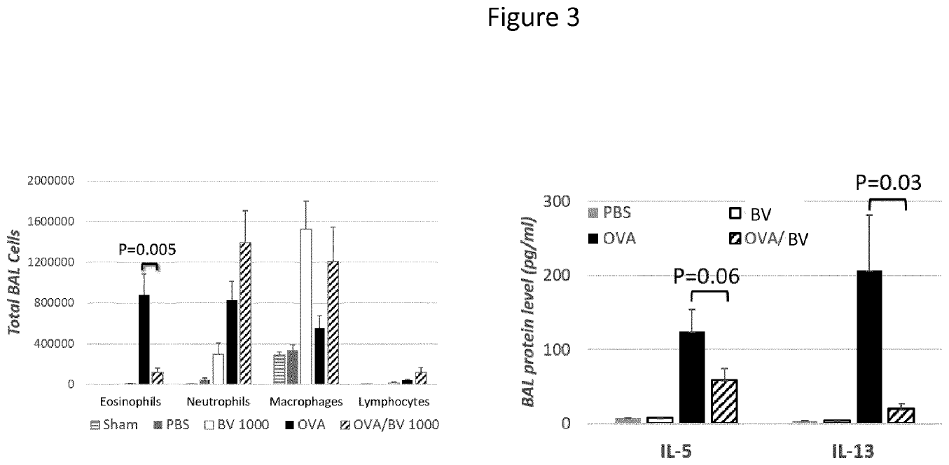

Identical set of data as in Example 2 but originating from a different experiment and where eosinophilia content from BAL was expressed as cell (FIG. 3A) and molecular content (FIG. 3B). As in Example 2, BV bacterial extract induced an inhibition of BAL eosinophilia with significant inhibition at 1 mg dose. Considering the high eosinophilia induction by OVA in cell numbers in the BAL (>800′000 cells), the almost complete and highly significant (P=0.005) abrogation of eosinophilia was unexpected for such product class.

### Example 4

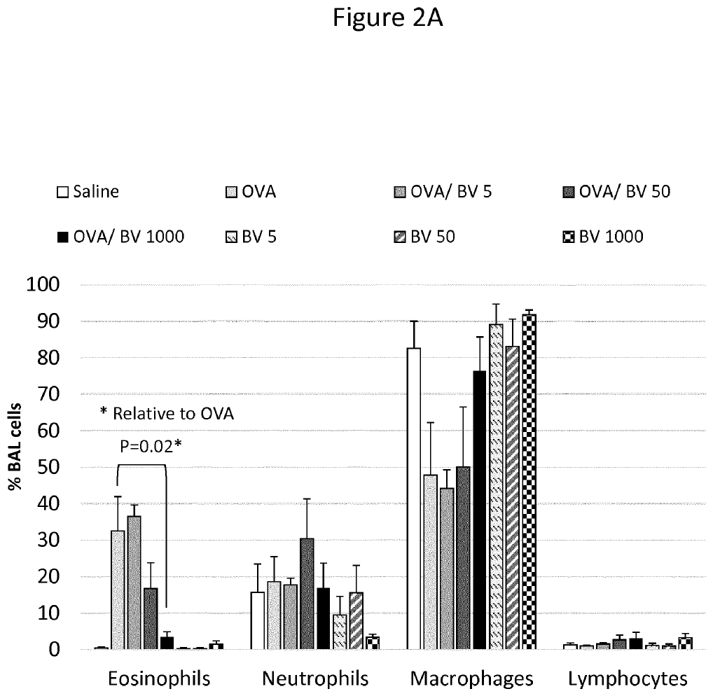

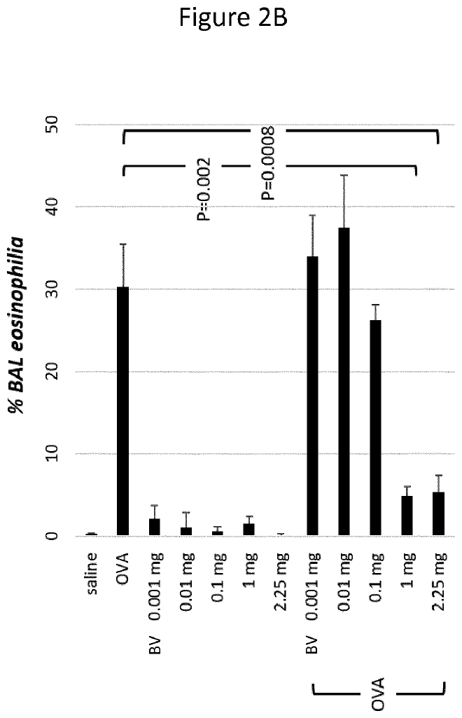

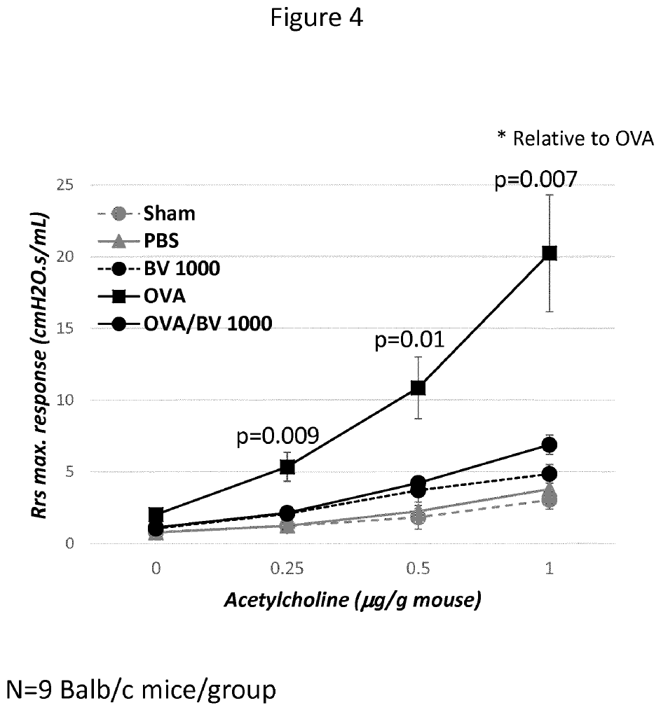

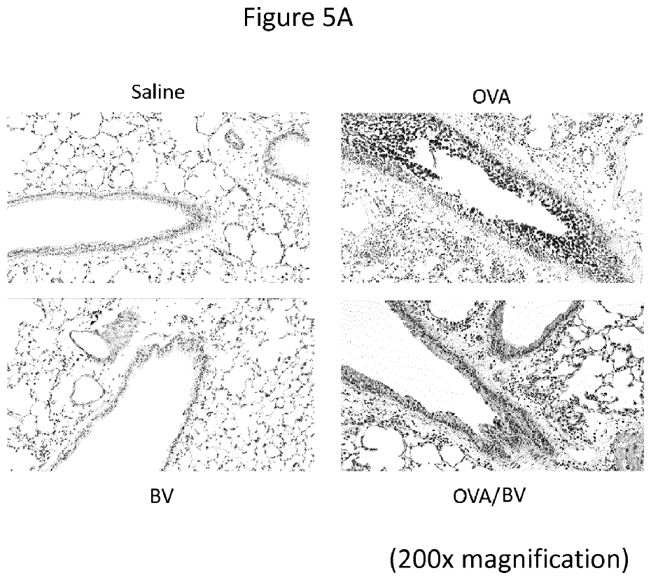

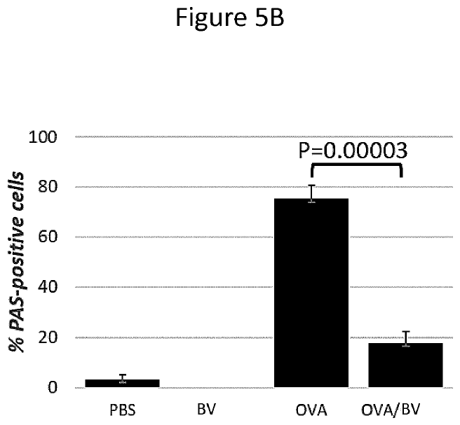

Representative example of two grouped experiments shown in FIG. 4 and demonstrating BV bacterial extract protection against AHR where best protective effects was obtained with 1 mg dose. The concomitant abrogation of eosinophilia shown in FIGS. 2 and 3, together with the functional shutdown of lung AHR demonstrated and validated the ability of intranasally administered BV bacterial extract to provide protection in a well-established model of allergic asthma, and show that a dose of 1 mg/day/mouse×14 treatments is adequate to confer optimal protection against allergen-driven AHR and airway inflammation. The protection achieved with this regimen was profound even though BV was administered to adult, rather than neonatal or young mice and its administration began simultaneously with, rather than before, exposure to the allergen. Further to this, the quasi absence of airway inflammation goblet cells from lung tissue in the OVA group treated with BV (OVA/BV) shown in FIG. 5A and quantitated in FIG. 5B as a highly significant decrease (P=0.00003) in Periodic acid-Schiff (PAS)⁺ mucus-secreting cells, functionally confirms the protection against AHR from FIG. 4.

### Example 5

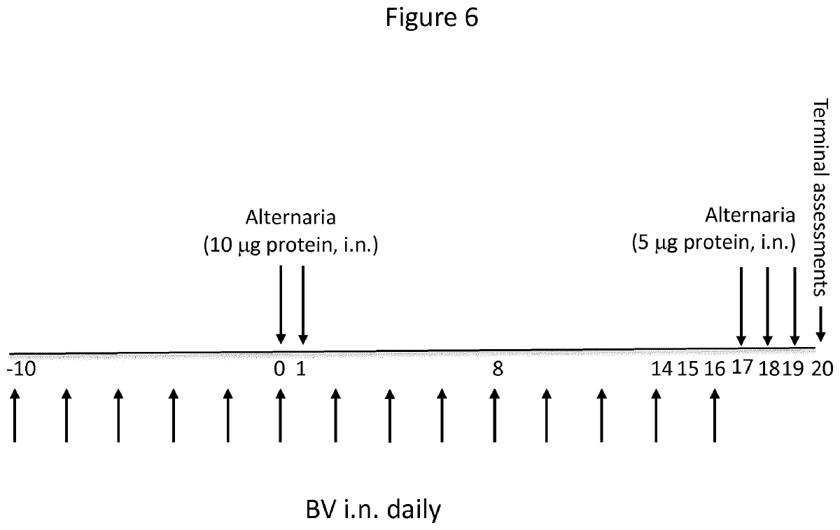

Scheme (FIG. 6) representing the experimental settings of intranasal (i.n.) BV bacterial extract in the Alternaria allergic asthma model using 1 mg dose daily following a regimen of 14 administrations as indicated.

### Example 6

Effects of i.n. BV bacterial extract in the Alternaria allergic asthma model monitored using BAL differentials.

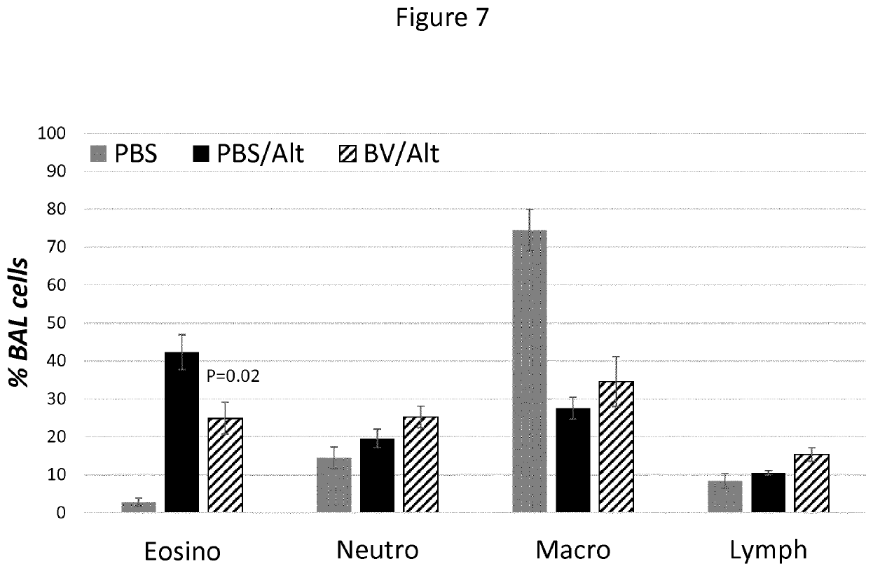

In the FIG. 7, approximatively 40% inhibition of eosinophilia in the BAL content was reached by BV bacterial extract using 1 mg dosage (14 times) as depicted in FIG. 6. Considering the high relevance of this human translatable model of asthma (same i.n. sensitization and challenge route without adjuvants), these results clearly showed the capacity of BV bacterial extract to confer protection in allergy-induced asthma.

BALB/c mice (4-6/group) were sensitized with Alternaria (Greer Laboratories: 50 μg of dry weight, 10 μg of protein in 50 μl of PBS) i.n. at day 0 and 1, and challenged i.n. with Alternaria (25 μg of dry weight, 5 μg of protein in 50 μl) at day 17, 18 and 19.

BV (concentrate, 1 mg in 50 μl (25 μl in each nostril) was administered every 2 days for 14 times from day −10. Terminal assessments were performed at day 20. BAL fluid was obtained by delivering cold 1% BSA in PBS (2 mL) into the airway via a tracheal cannula and gently aspirating the fluid. Cells were counted using a Countess II FL automated cell counter (Thermo Fisher Scientific) and differentials were determined by an operator blinded to mouse ID/grouping after staining with Hema 3 (Fischer) and examining at least 400 cells/slide.

### Example 7

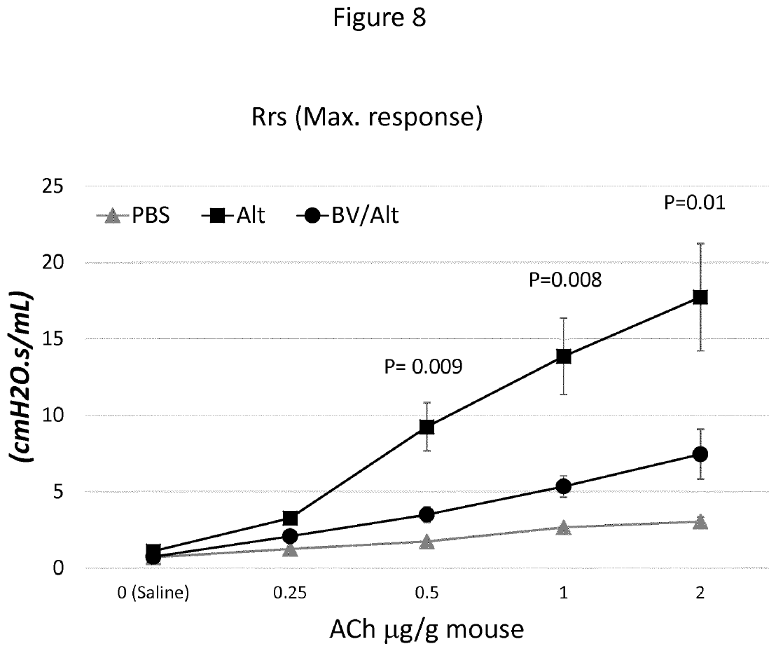

Shows a representative example of the functional efficacy correlation illustrated in the FIG. 8 with lung function as readout. Respiratory System Resistance (Rrs) response was significantly prevented by BV bacterial extract with best efficacy at 1 mg dose in all tested points. Significant reduction of AHR was expressed relative to Alternaria.

Airway resistance in response to increasing concentrations of acetylcholine (0-2 μg/g mouse) administered intravenously was assessed in animals anesthetized with ketamine and xylazine (100 and 10 mg/kg, respectively). Mice were tracheostomized, an 18-gauge metal cannula was inserted into the trachea and air leakage was prevented by a tightly tied suture thread. The tracheal cannula was connected to a computer-controlled ventilator for small animals (FlexiVent, Scireq Inc., Canada) and mice were mechanically ventilated with 150 breaths/min, tidal volume of 10 mL/kg, and positive end expiratory pressure (PEEP) of 3 cmH2O. Animals were placed on a warming pad to maintain body temperature and were paralyzed with pancuronium bromide (1 mg/kg, i.p.). In order to standardize lung volume history, the lungs were inflated twice to a pressure of 30 cmH2O (recruitment maneuvers). Acetylcholine chloride (ACh) diluted in saline at room temperature was injected in bolus through the right jugular vein in increasing doses of 0.25, 0.5, 1.0 and 2.0 μg/g body weight. Saline was used as a control. Immediately after each ACh dosing, measurements started and were performed every 30 seconds, over 5 min. The peak response after every dose of ACh was determined. Respiratory mechanics were evaluated using the constant phase model, which has the capacity to partition the respiratory properties into central and peripheral airways and to distinguish between different tissue properties. The parameters assessed were Newtonian resistance (Rn), a close approximation of resistance in the proximal conduction airways, and tissue damping (G), that is related to lung tissue resistance and reflects the energy dissipation in the alveoli. Only coefficient of determination (COD) equal or greater than 0.9 were used in the constant phase model.

As this was demonstrated in FIG. 7, the eosinophilia decrease of 30-40% in the BAL fully correlated with the functional readout of the lung where BV confirmed almost full protection against AHR in this extremely acute allergic asthma model.

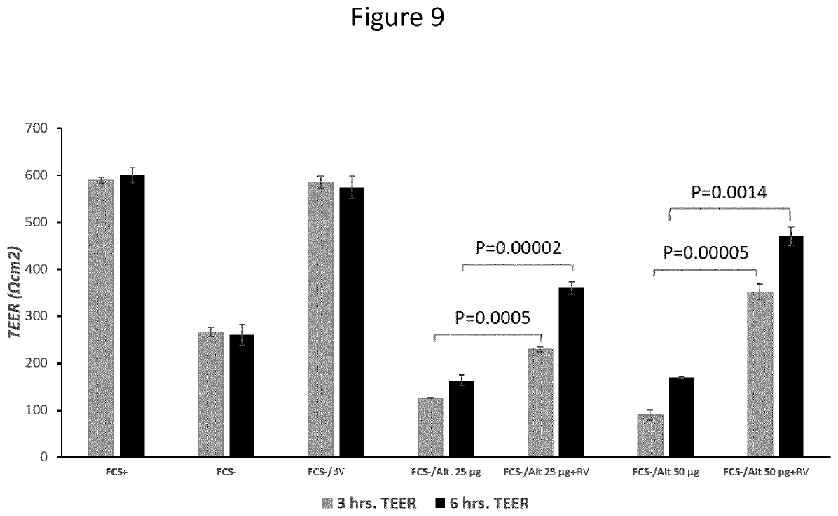

Further to this, monitoring the protective effect of BV on the tightness of the epithelial cell layer expressed as TEER (transepithelial electrical resistance), a measure of barrier function, shows the prevention by BV of stress- and Alternaria-induced impairment in human bronchial epithelial cells. This is demonstrated in FIG. 9 in an vitro model of airway cells enabling the study of drug transport using the 16HBE14o⁻ cell line (Ref: Forbes B, Int J Pharm. 2003, 257(1-2), 161-167)

### Example 8

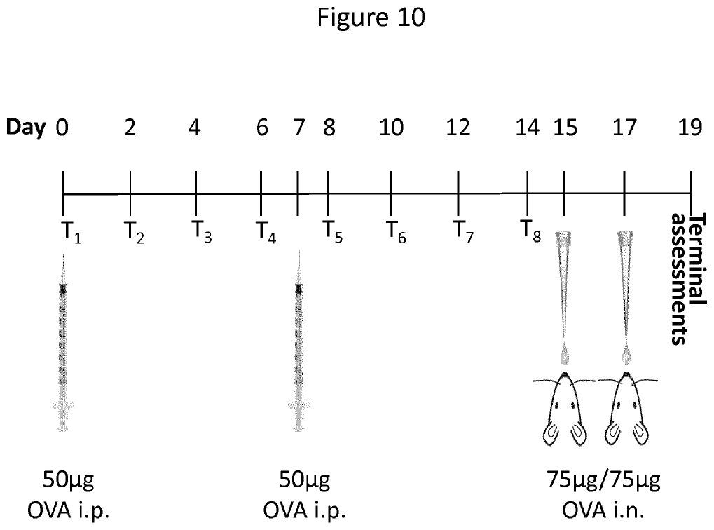

FIG. 10 shows a representative example of the protocol used in FIG. 1 but with changes in OVA-induced asthma. The model used again a preventive regimen of BV now reduced to 8 administrations (treatments T1 to T8), again followed by two consecutive OVA-induced sensitizations and challenges, with time reduction (Terminal assessment at day 19 instead of day 39). Significant eosinophilia blockade by BV was also confirmed in this new setting with fewer treatments.

### Example 9

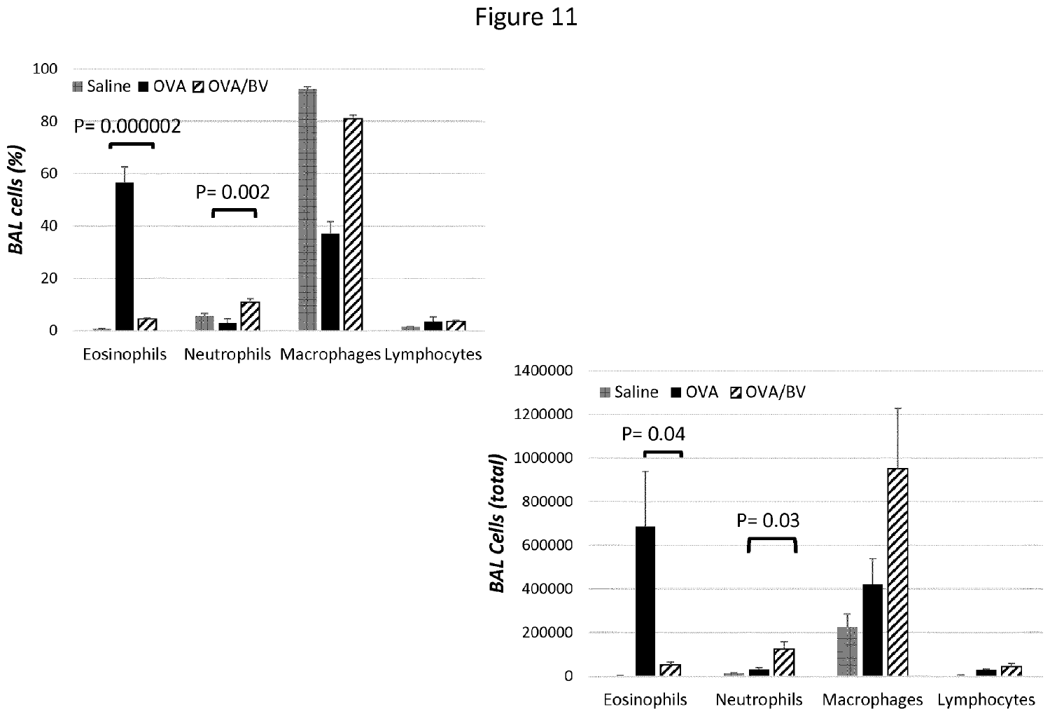

Shows the effects of BV on bronchoalveolar lavage (BAL) eosinophilia as in FIG. 2 expressed here as percentage (left) as well as cell count (right) but originating from the modified OVA-induced scheme depicted in FIG. 10. The FIG. 11 shows significant eosinophilia inhibition by BV bacterial extract, together with a significant increase of neutrophil recruitment in the BAL. Number of mice (n=4 mice/group) using 1 mg/50 μl.

### Example 10

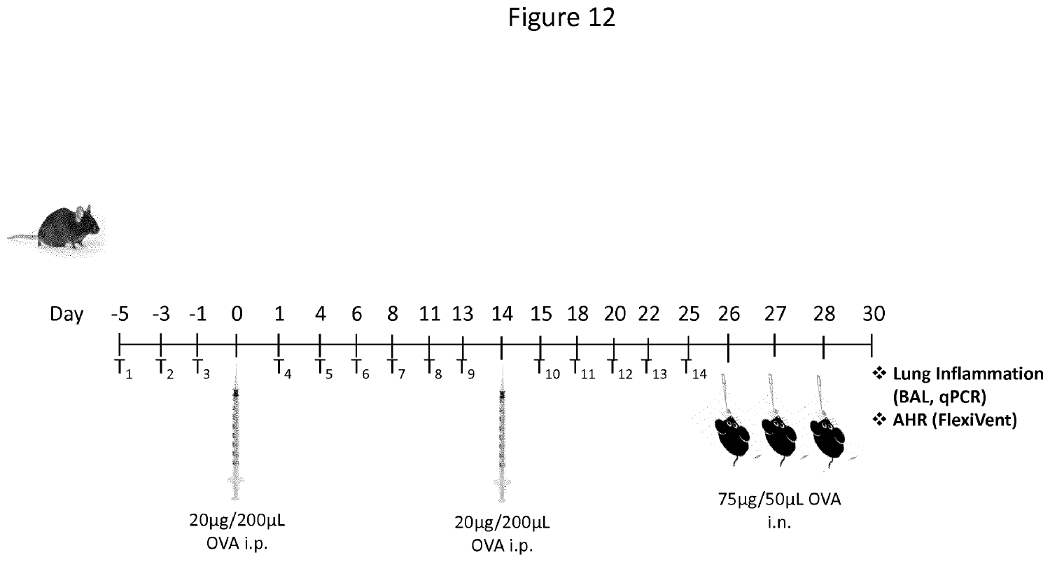

Assessment of OVA-induced asthma in a different mouse strain (C57BL6). FIG. 12 shows a representative example of the protocol used in FIG. 1 but with changes in OVA-induced asthma. The model again used a preventive regimen of BV bacterial extract using 14 (treatments T1 to T14) administrations followed by two consecutive OVA-induced sensitizations and three OVA challenges. Terminal assessment was at day 30.

### Example 11

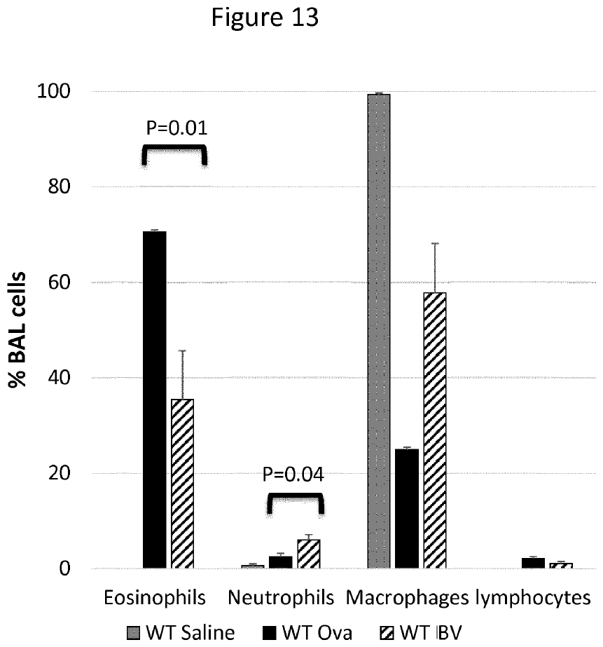

Example illustrating the effects of intranasal administration of BV bacterial extract in an OVA induced asthma model using the scheme depicted in FIG. 12 and adapted for C57 BL6 mice. Monitoring of efficacy was based on the BAL eosinophilia expressed in FIG. 13 as percentage of total BAL cells content (Y axis). Results showed the efficacy of BV in reducing eosinophilia associated with the protection of lung airway hyper responsiveness (AHR). Best efficacy in reducing eosinophilia was reached at a dose of 1 mg of BV.

### Example 12

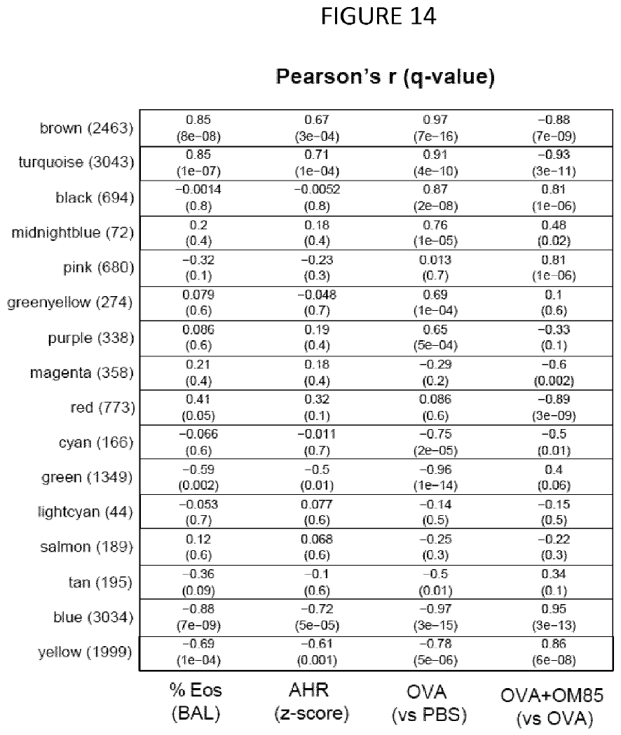

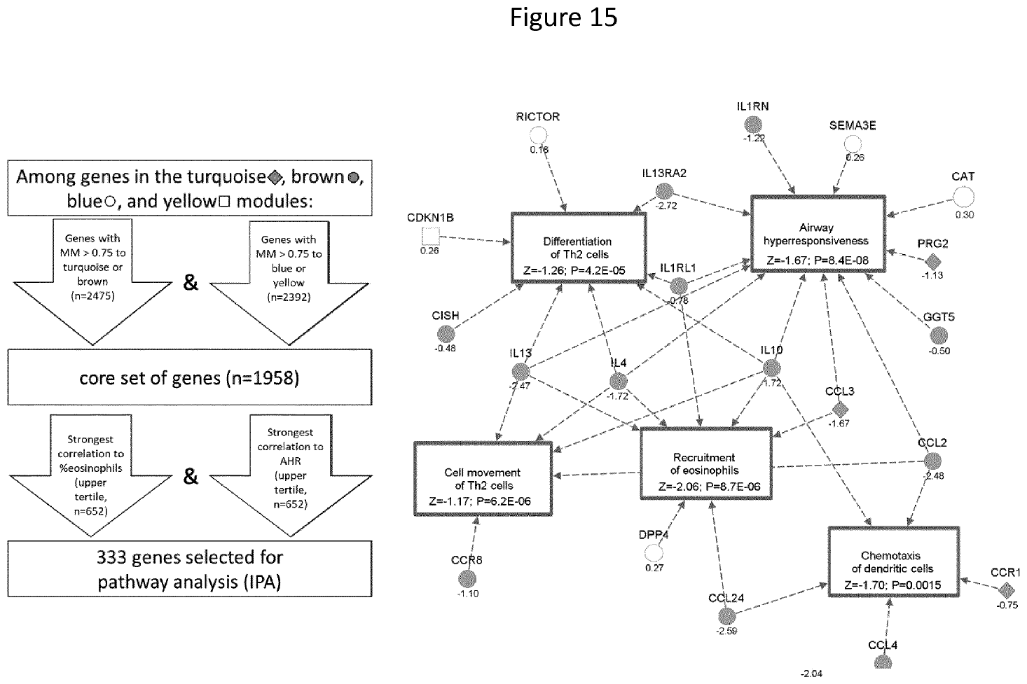

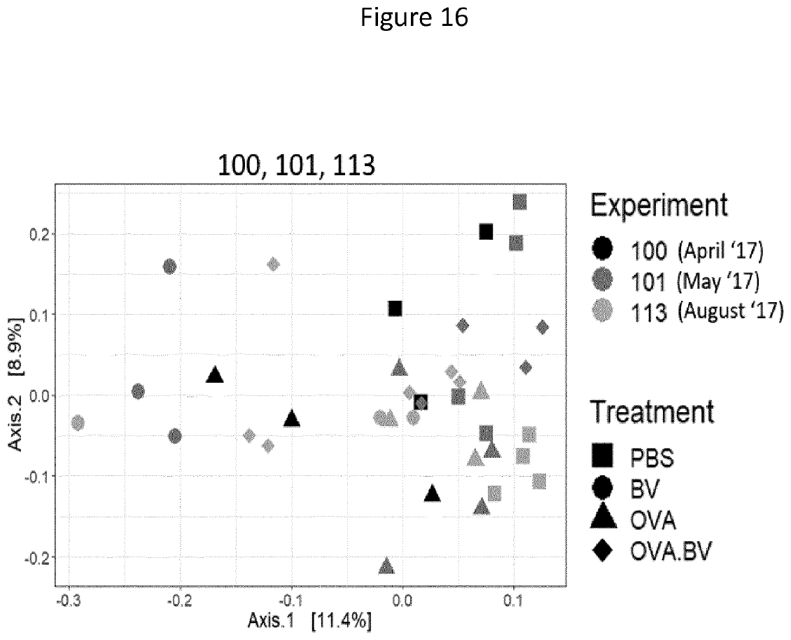

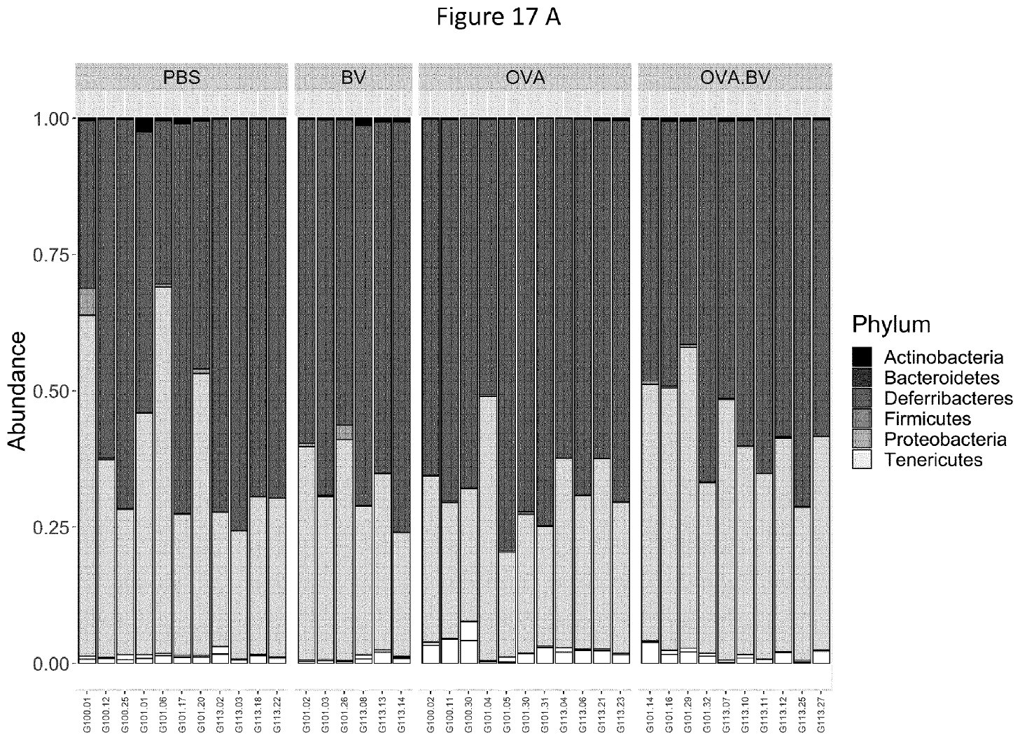

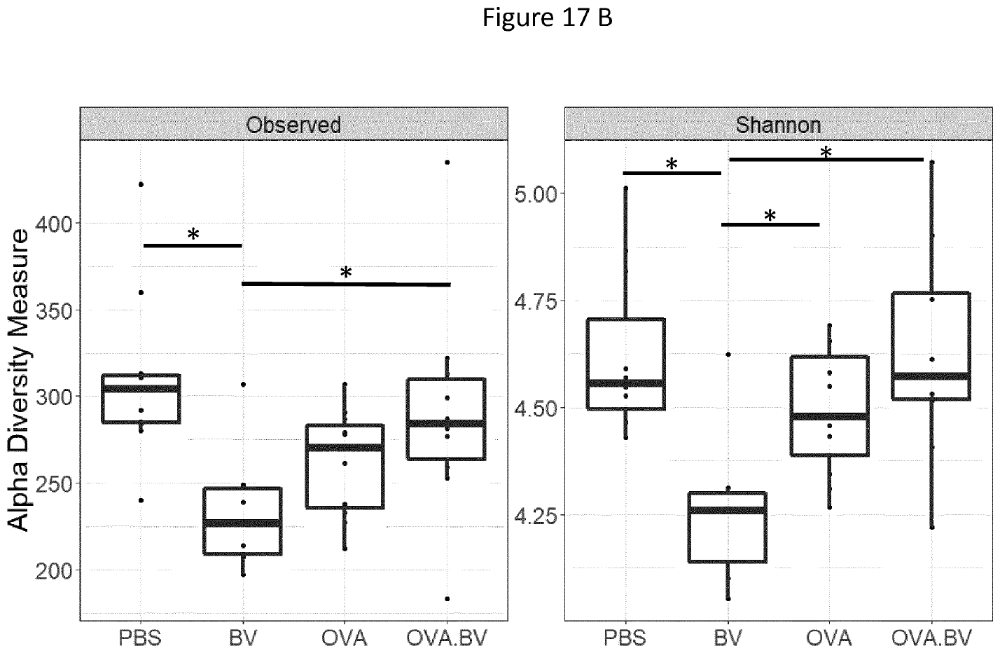

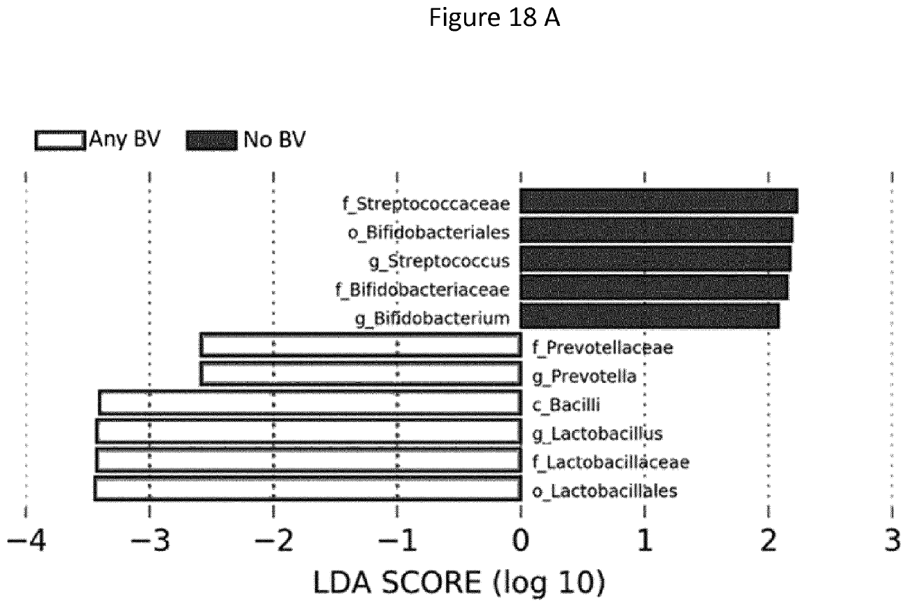

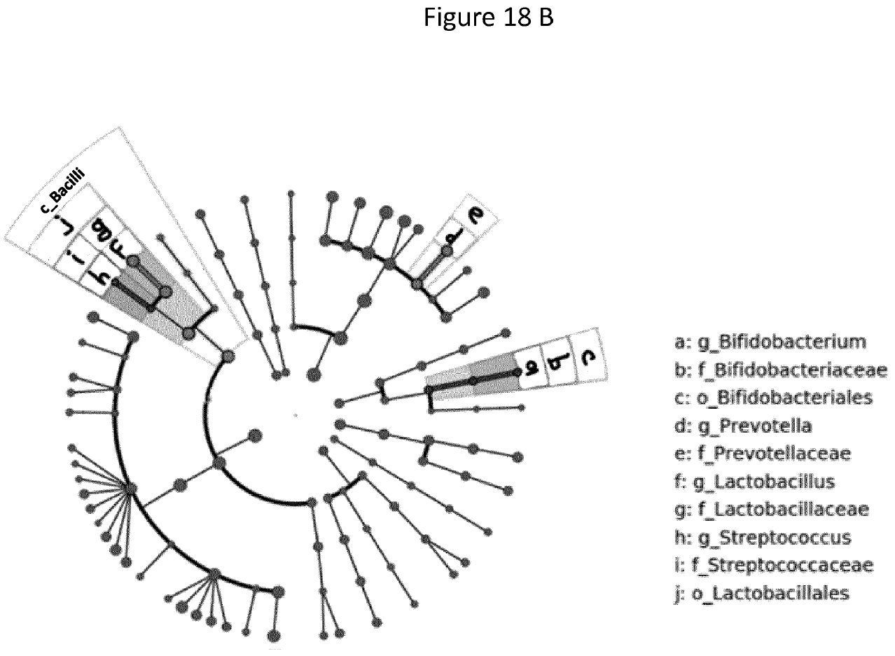

Core lung-expressed genes strongly associated with cardinal asthma phenotypes (AHR and BAL eosinophilia) in mice treated with OVA and/or BV cluster in pathway negatively associated with Th2 and dendritic cell migration as shown in FIG. 14.

Unfractionated lung tissue was collected from 27 Balb/c mice treated with PB, BV, OVA or OVA+BV as in FIG. 1, RNA was isolated, and RNA-Sequencing (RNA-Seq) was performed. Expression data were estimated for 24,538 genes and were filtered to include only genes with at least one read in 20% of samples, which retained a total of 19,613 genes. Weighted gene co-expression analysis (WGCNA) was performed on whole lung expression data for all 19,613 genes and co-regulated gene networks (modules) were constructed using the signed network algorithm. Expression data within each module were summarized using the module eigengene vector (i.e., the first principal component of the module), and the correlation to airway and immune phenotypes linked to protection was assessed using Pearson correlation. Ingenuity Pathway Analysis was used to determine enrichment for key biological terms or functions among differentially expressed function, where negative values correspond to predicted inhibition and positive values correspond to predicted activation.

WGCNA shown in FIG. 15 identified a total of 16 co-regulated gene networks (modules), only four of which were strongly and significantly associated with both eosinophilia and AHR (|Pearson's r|>0.5 and P≤0.001). The turquoise and brown modules were positively associated with AHR and eosinophilia and negatively with BV+OVA treatment (vs. OVA), whereas the blue and yellow modules showed negative associations with AHR and eosinophilia and positive association with BV+OVA treatment. Ingenuity Pathway Analysis (IPA) on a core set of genes from the brown, turquoise, blue, green and yellow modules that were highly associated with eosinophilia and AHR (n=333, FIG. 15 left) demonstrated that the genes perturbed by BV+OVA clustered not only, as expected, in pathways related to AHR and cell movement of eosinophils, but also in pathways related to migration of Th2 cells and dendritic cells (FIG. 15 right). Each of these pathways were predicted by IPA to be downregulated.

The DC-associated BV-induced transcriptional signatures identified by RNA-Seq prompted us to investigate the effects of the BV extract in vitro administration on bone marrow-derived DC (BMDC). Expression of MHC class II and costimulatory molecules (CD40, CD80 and CD86) was strongly inhibited in BV-treated BMDCs. We therefore asked whether DC reprogramming was sufficient to explain the suppressive effects of the BV bacterial extract on allergic asthma. To this purpose, BMDC pulsed with OVA in vitro for 2 days with or without a 2 day-pre-treatment with BV extract were transferred i.n. into naïve Balb/c mice that after 10 days were then challenged with OVA for three consecutive days. OVA-pulsed BMDC effectively elicited experimental asthma in these animals, as revealed by AHR, increased BAL eosinophilia and increased type-2 cytokine expression in the lungs and airway draining lymph nodes. Strikingly, BMDC preincubation with the BV bacterial extract was sufficient to strongly and significantly inhibit all of these allergen-driven responses, identifying DCs as a major target of BV-induced asthma protection in this model.

### Second Series of Experiments: Microbiota Changes

### Example 13

### Effects of Intranasal or Intratracheal BV Bacterial Extract on the Murine Gut Microbiome

In these experiments protective effects directly induced by BV bacterial extract were evidenced. This protective effect was mediated through changes in the gut microbiota of the tested mice. Intranasal and intratracheal administrations of BV bacterial extract in an asthma mouse model were showed to induce changes in the gut microbiome and is believed to contribute to decreased allergic responses.

Mice were treated intranasally with BV bacterial extract (5, 50, 1000 or 2500 microgram/treatment every 2 days) or PBS and sensitized and challenged with OVA or PBS. Fecal samples were collected for microbiome analysis.

As compared with PBS-only controls, a significant change in the gut microbial community structure was observed in mice receiving BV bacterial extract at 1000 or 2500 microgram/treatment, either with OVA or PBS (i.e. any high dose of BV bacterial extract) as indicated by beta diversity analysis (unweighted UniFrac) (FIG. 16). Furthermore, Lactobacillus was significantly enriched in any high dose of BV bacterial extract vs. PBS-only treated mice. These data clearly showed that BV bacterial extract induced significant changes in the murine gut microbiota.

### Example 14

### Effects on Gut Microbiome Profiles of Mice Treated Intranasally with High and Low Doses of BV Bacterial Extract

Mice were administered 14 intranasal doses (1 mg, or 2.25 mg) of BV bacterial extract. OVA sensitization occurred on Days 0 and 15, and OVA challenges on Days 28, 38, and 39. Control mice were administered PBS rather than OVA. At termination of experiment (Day 39), fecal pellets were collected. PBS- or OVA-challenged mice were treated with BV1000=1 mg dose, or with BV2250=2.25 mg dose. DNA was extracted from fecal pellets, representing the gut microbiome. Extracted DNA was sent to Argonne National Laboratory for 16S rRNA amplicon sequencing on the Illumina MiSeq. Sequence data were analyzed using QIIME2 and R. Linear discriminant analysis effect size (LEfSe) was performed to uncover differential features at multiple taxonomic levels. This analysis compared two groups of samples: Any BV (BV/OVA and BV only, regardless of the BV treatment dose) and No BV (OVA only and PBS only) within experiments 100, 101, and 113, using a cutoff of q<0.05 and LDA score>2 (FIG. 18). Phylogenetic relationships among differential features (g, genus; f, family; o, order; c, class) were also characterized FIG. 17).

These data clearly demonstrated that intranasal BV bacterial extract was capable of protecting from eosinophilia and AHR, and also led to a significant reorganization of the gut microbiome in protected mice, with an increase in taxa (especially Lactobacilli, FIGS. 18A and B) that are known to produce short chain fatty acids and to be associated with regulatory immunity/tolerance. Indeed, expansion of these bacteria is believed to contribute to the induction of the T regulatory cells we find in BV-treated, asthma-protected mice. Furthermore, results from the 16s rRNA sequencing experiments clearly evidenced a link between BV bacterial extract intranasal or intratracheal administration and immunomodulation.

1-24. (canceled)

25. A method of treating or preventing asthma, asthma exacerbation, allergic asthma conditions, wheezing associated symptoms, or microbiota associated respiratory disorders in a subject in need thereof, comprising administering to said subject a therapeutically effective amount of bacterial extract obtainable by alkaline lysis of Gram positive or Gram negative bacterial species.

26. The method of claim 25, wherein said Gram positive or Gram negative bacterial species are chosen from Moraxella catarrhalis, Haemophilus influenzae, Klebsiella pneumoniae, Staphylococcus aureus, Streptococcus pneumoniae, Streptococcus pyogenes, and Streptococcus sanguinis.

27. The method of claim 25, wherein said alkaline lysis is conducted at pH greater than 10.

28. The method of claim 25, wherein said bacterial extract is administered to said subject via the oral route.

29. The method of claim 25, wherein said bacterial extract is administered in the form of a solid or liquid.

30. The method of claim 29, wherein said bacterial extract is formulated as a pill, a tablet, a film tablet, a coated tablet, a capsule, syrup, powder or suppository.

31. The method of claim 25, wherein said bacterial extract is administered to said subject via intranasal or intratracheal route.

32. The method of claim 31, wherein said bacterial extract is administered to said subject at a dose ranging from 0.05 to 2 mg daily.

33. The method of claim 31, wherein said bacterial extract is administered to said subject at a dose ranging from 0.1 to 1 mg daily.

34. The method of claim 31, wherein said bacterial extract is administered to said subject at a dose ranging from 0.5 to 1 mg daily.

35. The method of claim 31, wherein said bacterial extract is administered to said subject at a dose around 1 mg daily.

36. The method of claim 31, wherein said bacterial extract is administered in the form of a solid, a liquid, or an aerosol.

37. The method of the claim 31, wherein said bacterial extract is formulated as a spray, droplet, colloidal, mist, nebulae, or atomized vapor.

38. The method of claim 31, wherein said bacterial extract is administered in the form of a powder, or crushable tablet.

39. The method of claim 31, wherein said bacterial extract is administered via a delivery device being selected from the group consisting of nasal insufflator device, intranasal inhaler, intranasal spray device, atomizer, nasal spray bottle, unit dose container, pump, dropper, squeeze bottle, nebulizer, metered dose inhaler (MDI), pressurized dose inhalers, insufflators, bi-directional devices, dose ampoules, nasal pads, nasal sponges, and nasal capsules.
